(19) United States
(12) Patent Application Publication
(10) Pub. No.: US 2026/0262730 A1
(43) Pub. Date: Sep. 10, 2026

(54) HEATING AND COOLING INHALATION DEVICE

(71) Applicant: Past Patterns, LLC, Santa Barbara, CA (US)

(72) Inventors: Davis KIYONAGA, Santa Barbara, CA (US); Evan ABEL, Rockaway Park, NY (US)

(21) Appl. No.: 19/550,650

(22) Filed: Feb. 26, 2026

Related U.S. Application Data
(60) Provisional application No. 63/766,708, filed on Mar. 4, 2025.

(51) Int. Cl. A24F 40/48; A24F 40/10; A24F 40/46; A24F 40/57; H05B 7/12

(52) U.S. Cl. CPC A24F 40/48; A24F 40/10; A24F 40/46; A24F 40/57; H05B 7/12

(57) ABSTRACT

An inhalation device includes a heating and cooling device, where the heating and cooling device extracts heat from a first side of the device and expels heat through a second side of the device. An airflow passage is thermally coupled to the first side of the heating and cooling device, while a container with vaporizable material is thermally coupled to the second side of the heating and cooling device. An atomizer can be included for thermally decomposing a substance within the container. The decomposed substance travels through the airflow passage, which can be configured to maximize heat extraction from the vaporized or decomposed substance. In some examples, the heating and cooling device may be a thermoelectric device. A fan can be used to force airflow across one or more thermal heat sinks, improving efficiency of the device.

### PRIORITY

This application claims the benefit of U.S. Provisional Patent Application No. 63/766,708, entitled “HEATING AND COOLING INHALATION DEVICE,” filed on Mar. 4, 2025, the entire disclosure of which is hereby incorporated by reference herein.

### BACKGROUND

Portable electronic inhalation devices rely on one or more thermal management components to vaporize a substance for inhalation. These devices typically include some type of heating element, whether it be a resistive coil, a ceramic heating element, or another thermally active component. Today's portable inhalation devices have various drawbacks.

For example, substances used in portable inhalation devices exhibit strongly temperature-dependent viscosity. At reduced ambient temperatures, the viscosity of such a substance rises substantially. Elevated viscosity restricts the flow of the substance toward the heating element. This restriction results in inadequate delivery of the substance to the atomizer, producing incomplete vaporization and a degraded user experience.

As another example, some portable inhalation devices attempt to address viscosity problems by using the atomizer heating element itself to pre-warm the substance reservoir. This approach raises thermal inefficiency concerns as well as substance degradation concerns. The atomizer heating element is designed for rapid, localized heating at the point of vaporization, not for uniform warming of the entire substance reservoir. The resulting uneven heat distribution causes portions of the substance in direct contact with the heating element to thermally degrade before the bulk of the substance reaches an adequate temperature.

As yet another example, the vaporization process necessarily produces vapor at elevated temperatures. Hot vapor traveling through the airflow passage of the device reaches the user's airway without an active reduction in temperature. Existing devices are not capable of actively cooling vapor within the device prior to inhalation.

As a result, a need exists for a portable inhalation device that addresses both the thermal conditioning of the substance reservoir and the temperature of the vapor delivered to the user, such as through an integrated thermal management approach that simultaneously warms the substance to a suitable viscosity for vaporization and reduces the temperature of the resulting vapor prior to inhalation, all while remaining compatible with the compact form factor of a portable device. Systems and methods disclosed herein address these needs.

### BRIEF DESCRIPTION OF THE DRAWINGS

FIG. 1 is a diagram illustrating the concept of thermoelectric heating and cooling in a manner suitable for use in the disclosed embodiments.

FIG. 2A is a cross-sectional view of an example inhalation device that includes heating the oil within an inhalation device and cooling airflow containing vaporized oil within the inhalation device, according to one or more embodiments herein.

FIG. 2B is a cross-sectional view of another example inhalation device that includes heating the oil within an inhalation device and cooling airflow containing vaporized oil within the inhalation device, according to one or more embodiments herein.

FIG. 2C is a cross-sectional view of another example inhalation device that includes heating the oil within an inhalation device and cooling airflow containing vaporized oil within the inhalation device, according to one or more embodiments herein.

FIG. 3 is a perspective view of an example inhalation device utilizing a heating and cooling device according to one or more embodiments herein.

FIG. 4 is a partially revealed perspective view of the example device of FIG. 3, according to one or more embodiments herein.

FIG. 5 is a cross-sectional view of the example device of FIGS. 3 and 4, according to one or more embodiments herein.

### DETAILED DESCRIPTION

Reference will now be made in detail to the present examples, including examples illustrated in the accompanying drawings.

The present disclosure relates to an inhalation device that vaporizes a substance and controls temperatures of the substance and the resulting vapor. An example inhalation device includes a heating and cooling device, where the heating and cooling device extracts heat from a first side of the device and expels heat through a second side of the device. The heating and cooling device can be a thermoelectric device, for example. An airflow passage is thermally coupled to the first side of the heating and cooling device, while a container with vaporizable material is thermally coupled to the second side of the heating and cooling device. An atomizer can be included for thermally decomposing a substance within the container. The decomposed substance travels through the airflow passage, which can be configured to maximize heat extraction from the vaporized or decomposed substance, such as by including a louvers or redirection vanes within a portion of the airflow passage. A fan can be used to force airflow across one or more thermal heat sinks, improving efficiency of the device.

The term “heating and cooling device” is used for convenience throughout this disclosure to refer non-exclusively to any device capable of simultaneously absorbing heat on one side and expelling heat on another side, including but not limited to thermoelectric devices based on the Peltier effect. The term “substance” is used for convenience throughout this disclosure to refer non-exclusively to any vaporizable or combustible material suitable for inhalation, including oils, nicotine-based liquids, cannabis-derived materials, flavored liquids, and other inhalable materials in liquid, semi-liquid, or solid form. The term “thermally decompose” is used for convenience throughout this disclosure to refer non-exclusively to any process by which a substance is converted from a liquid or solid state into a vapor or aerosol state through the application of heat, including vaporization and combustion. The term “airflow passage” is used for convenience throughout this disclosure to refer non-exclusively to any channel, conduit, or series of channels through which a vapor-air mixture travels within the inhalation device. These terms are not intended to be limiting in any way and are merely provided as accessible examples.

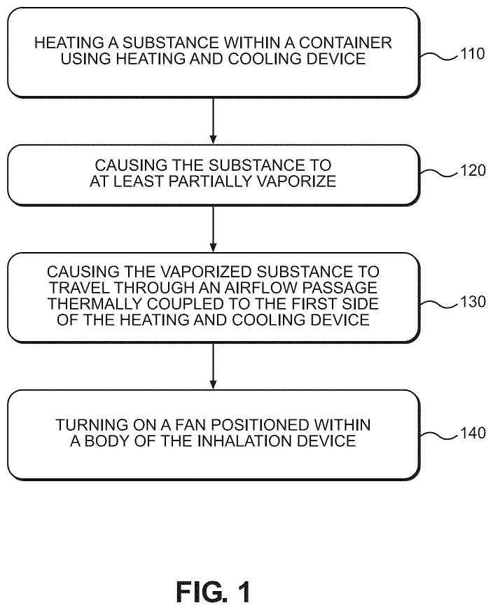

FIG. 1 illustrates a method for operating an inhalation device that manages thermal conditions during vaporization and inhalation. The method begins at step 110, where a heating and cooling device heats a substance contained within a container. This heating step warms the substance to reduce its viscosity, making it more suitable for vaporization. For example, oils and other viscous materials become easier to atomize when their temperature increases, leading to more consistent vapor production. In some examples, the heating and cooling device transfers thermal energy to the container through direct thermal coupling, such as through a shared wall or thermally conductive interface.

At step 120, the heated substance at least partially vaporizes. The vaporization can occur through an atomizer or other thermal decomposition mechanism that converts the substance from liquid or semi-liquid form into a vapor or aerosol. In an example, the atomizer applies localized heat to a portion of the substance, causing that portion to transition into the vapor phase. The resulting vapor mixes with ambient air drawn into the device, creating a vapor-air mixture suitable for inhalation. This vaporization process generates heat, raising the temperature of the vapor-air mixture above ambient temperature.

At step 130, the vaporized substance travels through an airflow passage that is thermally coupled to a first side of the heating and cooling device. As the vapor-air mixture flows through this passage, the heating and cooling device extracts heat from the mixture, cooling it before it reaches the user's mouth. For example, the first side of the heating and cooling device absorbs thermal energy from the vapor, lowering its temperature to a more comfortable level for inhalation. The extracted heat is simultaneously expelled through a second side of the heating and cooling device, which is thermally coupled to the container holding the substance. This dual-sided thermal behavior allows the device to cool the vapor while simultaneously warming the substance, addressing two thermal management challenges with a single component.

At step 140, a fan positioned within the body of the inhalation device turns on. The fan forces airflow across one or more heat sinks or thermal surfaces, improving the efficiency of heat transfer and enhancing the overall thermal management performance of the device. In some examples, the fan activates in response to a user input, such as pressing a button or initiating an inhalation cycle. In other examples, the fan operates continuously or cycles on and off based on temperature thresholds detected by sensors within the device.

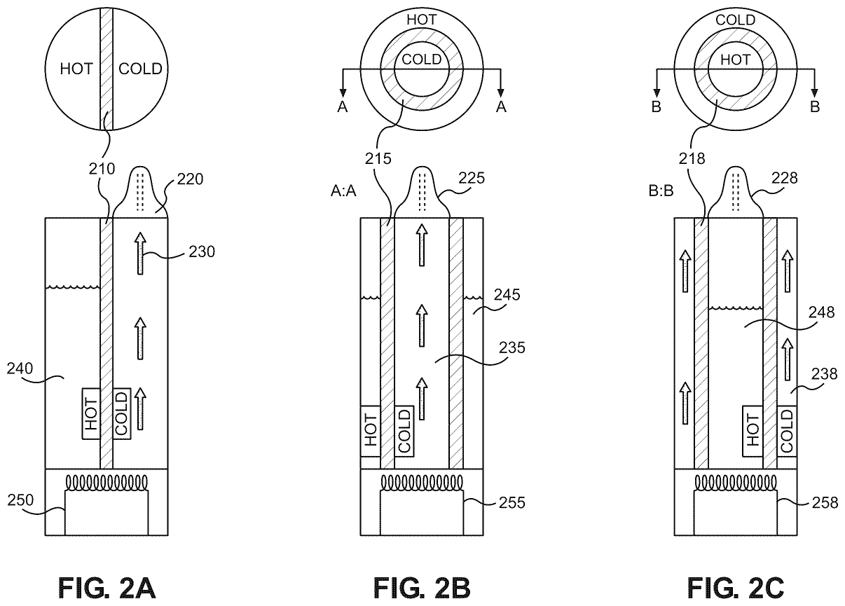

FIGS. 2A-2C provide cross-sectional views of several different conceptual inhalation devices that can practice the method of claim 1. As shown in the cross-sectional view of FIG. 2A, the heating and cooling device 210 can be oriented longitudinally along the center of the inhalation device, dividing the device into a hot side and a cold side. The heating and cooling device 210 is oriented such that the hot side is in thermal communication with a container 240 for oil or other liquid to be vaporized. By heating the oil 240, proper vaporization and operation of the inhalation device is ensured.

Meanwhile, the cooling side of the heating and cooling device 210 is in thermal communication with an airflow passage 230. When a user inhales through the mouthpiece 220, air is pulled through an atomizer 250 that heats the air and mixes it with a portion of the oil in the container 240 to vaporize that portion of oil. The vaporized oil, mixed with the heated air, then travels through the airflow passage 230. The heating and cooling device 210 absorbs heat from the heated air, cooling it to provide a more pleasant experience to the user.

FIG. 2B provides another example inhalation device. As shown in the cross-sectional views of FIG. 2B, the example inhalation device utilizes a heating and cooling device 215 with an annular cross section. The heating and cooling device 215 can include a central axis that extends through the center of the annular cross section and is aligned with a longitudinal axis of the inhalation device itself. This alignment allows the center of the heating and cooling device 215 to align with the direction of airflow 235 through the device 215.

As shown in the drawing, the outer surface of the heating and cooling device 215 can be in thermal communication with a container 245 for oil or liquid. In this example, the container 245 surrounds the heating and cooling device 215. The outer surface of the heating and cooling device 215 is arranged as the heating side, while the inner surface of the heating and cooling device 215 is arranged as the cooling side.

In operation, a user inhales through the mouthpiece 225, causing air to enter the atomizer 255 and become heated for vaporizing a portion of the contents of the container 245. The air heated by the atomizer 255 vaporizes the portion of the contents in the container 245, and the heated airflow with vaporized content then travels through the airflow passage 235, cooling down based on thermal communication with the cold inner surface of the heating and cooling device 215.

In another embodiment depicted in FIG. 2C, a heating and cooling device 218 with an annular cross section can be used, but with the hot and cold sides reversed with respect to FIG. 2B. That is, the heating and cooling device 218 can be configured such that an interior surface of the heating and cooling device 218 expels heat, while an outer surface of the heating and cooling device 218 absorbs heat. This is a reverse configuration relative to what is shown in FIG. 2B. In this example configuration, the oil container 248 is located on the inside of the heating and cooling device 218, such that it receives heat from the inner surface of the heating and cooling device 218. Meanwhile, the airflow of vaporized or combusted material flows in the outer portion 238, such that the outer surface of the heating and cooling device cools the airflow before it exits through the mouthpiece 228.

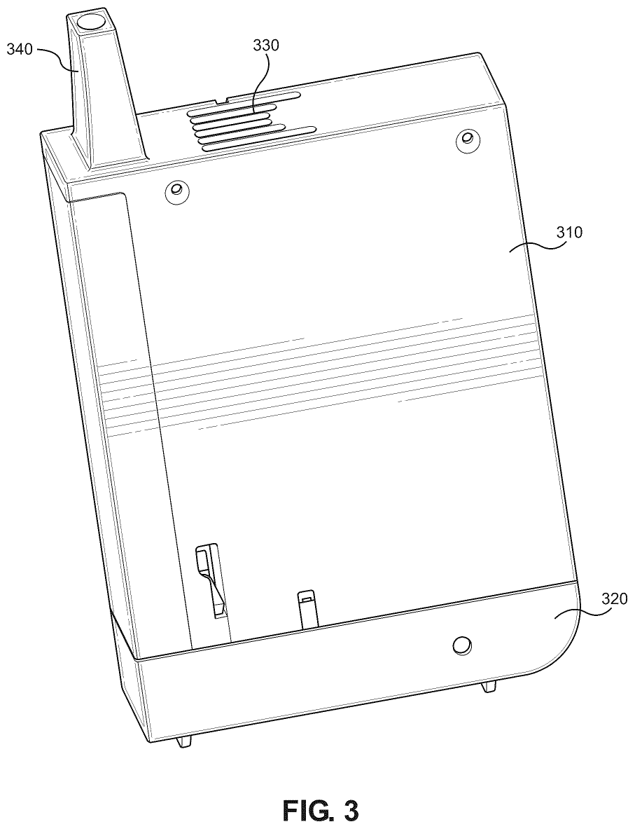

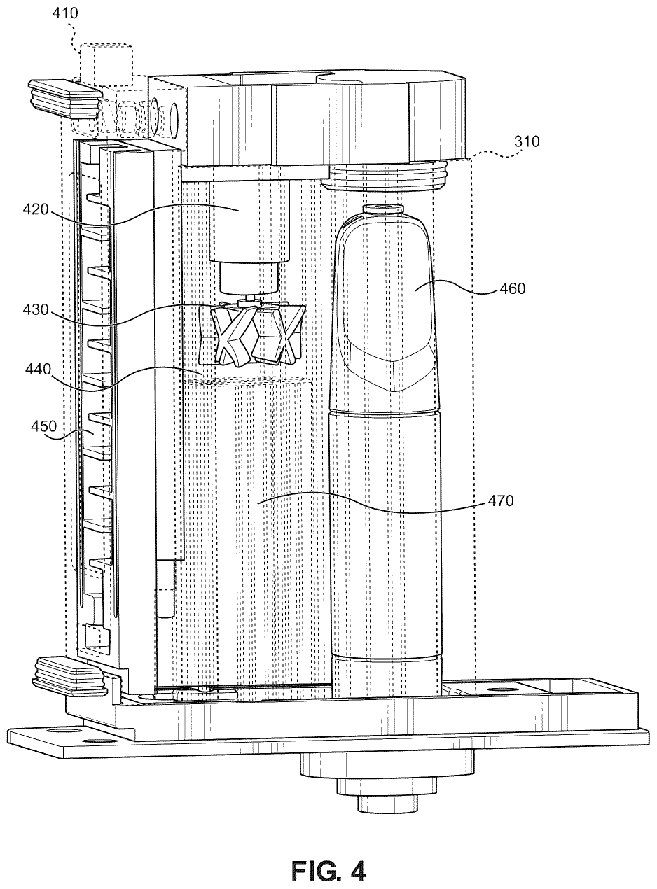

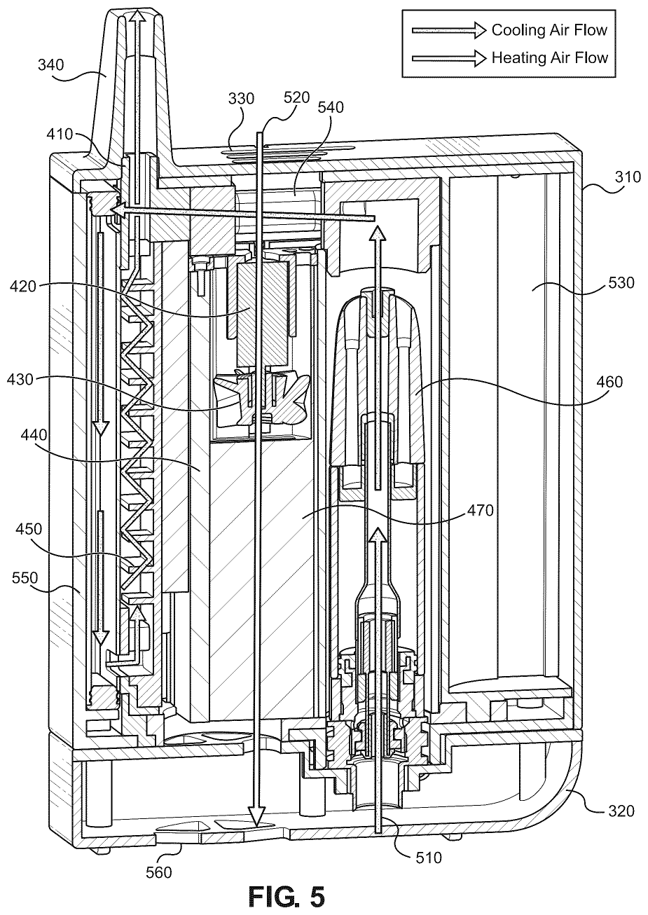

FIG. 3 illustrates an external perspective view of an inhalation device according to one or more examples herein. The view shows the outer housing and user-accessible features of the device, while the internal thermal management components and airflow structures are housed within and are described in greater detail in relation to FIGS. 4 and 5. The device integrates heating, cooling, vaporization, and airflow control functions within a compact, portable form factor suitable for handheld operation.

As shown in FIG. 3, the inhalation device features a body portion 310 that forms the primary outer housing. The body portion 310 defines a generally rectangular enclosure with rounded edges, providing structural protection for the internal components while establishing the overall form factor of the device. A lower body portion 320 is situated at the base of the body portion 310, forming the bottom region of the housing. This lower region features a curved corner profile that transitions smoothly from the vertical sidewalls to the base, creating a stable resting surface when the device is placed on a flat surface. For example, support projections or feet elevate the device slightly above the resting surface in some configurations, allowing air to circulate beneath the device and facilitating heat dissipation from internal components.

Near the top edge of the body portion 310, a set of air vents 330 is formed as a group of short, parallel, raised or recessed bars extending across a portion of the top surface, although other configurations can be used. These air vents 330 permit air to pass into or out of the interior of the body portion 310, facilitating convective airflow that assists in dissipating heat generated by internal thermal management components. In an example, heated air expelled from a second side of a heating and cooling device (described in relation to FIGS. 4 and 5) exits the body portion 310 through these vents, thereby maintaining a temperature differential across the heating and cooling device and improving overall thermal efficiency. The air vents 330 are oriented to direct expelled air away from the mouthpiece 340 in some configurations, preventing warm exhaust air from mixing with the cooled vapor stream that exits through the mouthpiece 340.

The mouthpiece 340 extends upward from the top surface of the body portion 310 as a tower-like protrusion with a generally rectangular vertical body and a circular opening at its top. The mouthpiece 340 is positioned near a corner of the body portion 310, providing an ergonomic exit port through which a user inhales the cooled vapor-air mixture. The circular opening at the top of the mouthpiece 340 terminates the airflow passage within the device, delivering the processed vapor-air mixture to the user's mouth. Internal contouring or tapering guides the vapor-air mixture smoothly from the internal airflow passage to the exit opening in some examples, reducing turbulence and improving the user experience.

The configuration depicted in FIG. 3 provides a compact and portable form factor suitable for handheld operation. The external housing encloses the internal thermal management components, airflow passages, and vaporization mechanisms that are described in greater detail in relation to FIGS. 4 and 5. By integrating heating, cooling, and airflow control functions within a single housing, the device addresses multiple thermal management challenges while maintaining a user-friendly design. In some examples, the body portion 310 is constructed from thermally insulating materials that reduce heat transfer to the user's hand during operation, improving comfort and safety. In other examples, internal mounting structures or brackets secure the internal components in fixed positions, ensuring consistent thermal coupling and airflow performance across repeated use cycles.

FIG. 4 illustrates an internal assembly view of the inhalation device, revealing the thermal management components, airflow structures, and vaporization mechanisms housed within the body portion 310 described in relation to FIG. 3. The view shows the device with the outer housing partially removed or rendered transparent, exposing the internal architecture that performs the heating, cooling, and vapor conditioning functions. The assembly is mounted on a base plate that supports and aligns the various components, ensuring proper thermal coupling and airflow paths throughout the device.

A left vertical support structure extends upward from the base plate, featuring a series of stacked or integrated components labeled 420, 430, 440, and 450. Element 420 is a fan motor positioned near the top of the assembly, providing mechanical power to drive airflow through the device. Below the fan motor 420, element 430 includes fan blades that rotate when driven by the motor 420, forcing air through adjacent passages and across thermal surfaces. The fan blades 430 accelerate ambient air drawn into the device, increasing convective heat transfer and improving the efficiency of thermal management. Element 440 is a heating and cooling device positioned alongside the fan blades 430, featuring a first side that absorbs heat and a second side that expels heat. For example, the heating and cooling device 440 operates based on the Peltier effect, transferring thermal energy from one side to the other when electrical current flows through the device. Element 450 is a baffled airflow passage situated above the heating and cooling device 440, defining a channel through which the vapor-air mixture travels. The baffles within passage 450 redirect the airflow, increasing turbulence and maximizing thermal contact between the vapor-air mixture and the cooling surfaces of the heating and cooling device 440.

Element 440 is a heating and cooling device positioned alongside the fan blades 430, featuring a first side that absorbs heat and a second side that expels heat. The heating and cooling device 440 can extend vertically as shown in more detail with respect to FIG. 5. The device 440 can be configured such that the cooling side of the device 440 is thermally coupled to an airflow passage 450, while the heating side of the device is thermally coupled to the fan, a heat sink, and/or a container. In one example, the heating and cooling device 440 operates based on the Peltier effect, transferring thermal energy from one side to the other when electrical current flows through the device.

Element 450 is a baffled airflow passage situated alongside the heating and cooling device 440, defining a channel through which the vapor-air mixture travels. The baffles within passage 450 redirect the airflow, increasing turbulence and maximizing thermal contact between the vapor-air mixture and the cooling surfaces of the heating and cooling device 440.

Above the baffled airflow passage 450, a mouthpiece flow tube 410 is positioned, represented by dashed lines in the drawing. The dashed rendering of element 410 indicates that it is either partially transparent, removable, or positioned behind other components in the perspective view. The mouthpiece flow tube 410 connects the baffled airflow passage 450 to the mouthpiece 340 (described in relation to FIG. 3), providing a conduit through which the cooled vapor-air mixture travels from the thermal management assembly to the user's mouth. In some examples, the vertical alignment of elements 410 through 450 creates a continuous airflow pathway that guides the vapor-air mixture from the vaporization zone, through the thermal conditioning zone, and finally to the exit port at the mouthpiece 340.

Within the right-side envelope of the body portion 310, a container 460 is visible, holding the substance to be vaporized. The container 460 stores oil, nicotine-based liquid, cannabis-derived material, or other inhalable substance in liquid or semi-liquid form. In some examples, the container 460 incorporates a threaded closure, snap-fit cap, or elastomeric seal that allows the user to refill the container 460 with fresh substance.

Extending between the hot side of the heating and cooling device 440 and the container 460 are thermal coupling fins 470, which increase the surface area available for heat transfer between the heating and cooling device 440 and the substance within the container 460. The thermal coupling fins 470 conduct heat from the second side of the heating and cooling device 440 to the substance, warming the substance and reducing its viscosity to facilitate vaporization. For example, the fins 470 are constructed from thermally conductive materials such as aluminum, copper, or other metals that efficiently transfer thermal energy. By increasing the contact area between the heating source and the substance, the thermal coupling fins 470 improve the rate and uniformity of heating, ensuring consistent vaporization performance.

The heating and cooling device 440 is positioned such that its first side (the cooling side) is thermally coupled to the baffled airflow passage 450, extracting heat from the vapor-air mixture as it flows through the passage. The second side of the heating and cooling device 440 (the heating side) expels the extracted heat, and this heating side is thermally coupled to one or more heat dissipation structures. In some examples, the heating side couples to the thermal coupling fins 470.

In other examples, the heating side couples to the fan motor 420, fan blades 430, or a dedicated heat sink, dissipating the extracted heat into the ambient air forced through the device by the fan. In still other examples, the heating side couples to multiple structures simultaneously, transferring heat to both the container 460 and the fan assembly, thereby distributing thermal energy across multiple pathways and optimizing overall thermal management. The internal elements also include mounting brackets, thermal insulators, and sealing gaskets that maintain proper alignment and prevent unwanted heat transfer between hot and cold regions. By positioning these elements between the left assembly and the container 460, the device achieves efficient thermal management while maintaining a compact overall footprint.

The internal assembly depicted in FIG. 4 integrates multiple thermal management functions within a unified structure. The left vertical assembly (elements 410-450) provides airflow control, thermal conditioning, and vapor delivery, while the container 460 and thermal coupling fins 470 store and thermally condition the substance for vaporization. The fan motor 420 and fan blades 430 force air through the device and across the thermal fins 470, improving convective heat transfer across the heating and cooling device 440. The baffled airflow passage 450 maximizes thermal contact between the vapor-air mixture and the cooling side of the heating and cooling device 440, while the thermal coupling fins 470 maximize thermal contact between the heating side of the device 440 and the substance within the container 460, as well as the air moved by the fan 420.

FIG. 5 illustrates a sectional view of the inhalation device, revealing the internal airflow paths, thermal interfaces, and component arrangements within the body portion 310 described in relation to FIGS. 3 and 4. The sectional view exposes the vertical stacking of internal components and the routing of two distinct airflow streams through the device, distinguished in the figure by a legend identifying a cooling air flow path 510 and a heating air flow path 520. These two airflow paths can operate simultaneously within the device in some examples, each serving a distinct thermal management function. In other examples, however, either of the airflow paths can operate alone. In other words, the device can be configured to performing cooling only, heating only, or both heating and cooling simultaneously.

The outer housing visible in FIG. 5 may include the body portion 310 forming the right sidewall and the lower body portion 320 forming the rounded lower right corner, consistent with the external features described in relation to FIG. 3. The top surface of the housing may include an internal upper plate or partition corresponding to the air vents 330, extending across the upper width of the device interior. On the left side, the housing narrows upward into a tapered region corresponding to the mouthpiece 340, through which the cooled vapor-air mixture exits the device toward the user.

The sectional view of FIG. 5 reveals a horizontal layering of components moving from the left (outer) side to the right (inner) side of the device. The airflow passage wall 550 forms the outermost boundary on the left side, defining an outer passage through which the vapor-air mixture travels downward as part of the cooling air flow path 510. The inner wall of this outer passage serves simultaneously as the outer wall of the baffled airflow passage 450, creating a shared boundary between the two flow channels. The opposite side of the baffled airflow passage 450 is thermally coupled to the first side (cooling side) of the heating and cooling device 440, allowing the device 440 to extract heat from the vapor-air mixture as it flows through the baffled passage.

The container 460 occupies the region immediately to the right of the heating chamber, with one side of the container 460 exposed to the thermal coupling fins 470 and the heated airflow within the chamber. This thermal coupling warms the substance within the container 460, reducing its viscosity and facilitating vaporization at the base of the container 460. Beyond the container 460, the battery compartment 530 occupies the rightmost region of the device, providing space for a battery that supplies electrical power to the fan motor 420, the heating and cooling device 440, and other active components. The battery compartment 530 is separated from the thermal management components by the container 460, reducing heat transfer to the battery and improving safety and performance.

Within the heating chamber, several components are aligned vertically from top to bottom, forming a vertical stack through which the heating air flow path 520 travels. The air vents 330 occupy the uppermost position, admitting ambient air into the heating chamber. Below the air vents 330, the fan motor 420 drives the fan blades 430, which force air downward through the chamber and across the thermal coupling fins 470. The thermal coupling fins 470 occupy the central vertical region, extracting heat from the heating side of device 440 and warming the container 460. At the base of this vertical stack, the lower air vents 560 allow the heated air to exit the heating chamber, expelling thermal energy into the surrounding environment. In some examples, the vertical alignment of these components minimizes the horizontal footprint of the heating chamber, allowing the device to maintain a compact overall form factor while still providing sufficient airflow and heat transfer capacity.

Moving further inward from the cooling side just described, the second side (heating side) of the heating and cooling device 440 faces a heating chamber that contains the fan motor 420, fan blades 430, and thermal coupling fins 470. This heating chamber provides the space through which the heating air flow path 520 travels, carrying thermal energy away from the heating side of device 440. The thermal coupling fins 470 extend horizontally across the chamber from the heating side of device 440 toward the container 460, which forms the opposite wall of the heating chamber. For example, the fins 470 increase the surface area available for convective heat transfer, improving the efficiency with which the fan-driven airflow extracts heat from the heating side of device 440 and delivers it to the container 460 or outside the device entirely.

The heating air flow path 520 is represented by flow arrows in the sectional view, indicating the general direction of air movement through the heating chamber. As described above, ambient air enters through the air vents 330, is forced by the fan blades 430 across the thermal coupling fins 470, extracts heat from the heating side of device 440, and continues downward to the lower chamber near the base of the container 460. In this lower chamber, the heated air mixes with ambient air entering through the lower air vents 560, warming the base of the container 460 to facilitate vaporization before expelling through the lower air vents 560 and dissipating thermal energy into the surrounding environment.

The cooling air flow path 510 is represented by flow arrows in the sectional view, indicating the general path of the vapor-air mixture from the vaporization zone to the mouthpiece 340. Vaporization occurs at the base of the container 460, where an atomizer or heating element (not separately labeled) thermally decomposes the substance. The resulting vapor-air mixture exits the top of the container 460 and enters the intermediate air passage 540, which is oriented horizontally along an upper section of the device. The intermediate air passage 540 is positioned above the fan motor 420 and fan blades 430 but does not occupy the entire upper width of the device, allowing heated air from the heating air flow path 520 to travel downward through the air vents 330 and into the fan chamber.

From the intermediate air passage 540, the vapor-air mixture travels downward along the airflow passage wall 550, which forms the outer boundary of the cooling air flow path on the left side of the device. The vapor-air mixture then turns upward and enters the baffled airflow passage 450, where the first side (cooling side) of the heating and cooling device 440 extracts heat from the mixture. In some examples, the cooling side of the heating and cooling device 440 is thermally coupled to the intermediate air passage 540, the airflow passage wall 550, the baffled airflow passage 450, or multiple portions of the cooling air flow path 510 simultaneously, maximizing heat extraction and reducing the temperature of the vapor-air mixture before it reaches the user's mouth. The cooled vapor-air mixture continues upward through the mouthpiece flow tube 410 and exits through the mouthpiece 340.

The sectional view of FIG. 5 illustrates how the cooling air flow path 510 and the heating air flow path 520 operate in parallel within the device without thermally interfering with one another. The two airflow streams can be physically separated, ensuring that the heated air expelled from the heating chamber does not mix with the cooled vapor-air mixture traveling toward the mouthpiece 340. In an example, the airflow passage wall 550 is constructed from a thermally insulating material that reduces conductive heat transfer between the two adjacent airflow channels, maintaining the temperature differential that enables effective thermal management.

The arrangement depicted in FIG. 5 allows the device to simultaneously cool the vapor-air mixture for comfortable inhalation and expel waste heat through the lower air vents 560, addressing both thermal management functions within a single compact assembly. By routing the two airflow paths through distinct channels separated by the airflow passage wall 550, the device achieves efficient thermal conditioning without requiring separate housings or complex ducting systems.

The specific configuration depicted in FIGS. 3-5 represents one example arrangement of components within an inhalation device. Other configurations are possible and fall within the scope of this disclosure. For example, the heating and cooling device 440 need not be positioned alongside the fan assembly in a horizontal arrangement. In some examples, the heating and cooling device is positioned above or below the fan assembly in a vertical stack, with the cooling side thermally coupled to an airflow passage that wraps around or passes through the device. In other examples, the heating and cooling device is oriented at an angle relative to the primary airflow direction, allowing the cooling side to extract heat from multiple portions of the cooling air flow path simultaneously while the heating side expels heat into a dedicated exhaust chamber.

The thermal coupling fins 470 shown in FIGS. 4-5 extend horizontally across the heating chamber, but alternative fin configurations are contemplated. In some examples, the fins extend vertically, creating a vertical array of heat transfer surfaces through which the fan-driven airflow travels. In other examples, the fins are arranged in a radial pattern around a central axis, maximizing surface area within a cylindrical or annular heating chamber. The fins need not be planar; curved, corrugated, or pin-fin geometries increase turbulence and improve convective heat transfer in some configurations. In still other examples, the thermal coupling between the heating side of device 440 and the container 460 is achieved without discrete fins, using instead a thermally conductive plate, heat pipe, or direct contact interface.

The airflow paths depicted in FIG. 5 show the cooling air flow path 510 traveling downward along the outer wall 550 before turning upward into the baffled passage 450. Alternative routing is possible. In an example, the vapor-air mixture exits the container 460 and travels directly into the baffled airflow passage 450 without first traveling downward along an outer wall, shortening the overall flow path and reducing pressure drop. In another example, the cooling air flow path includes multiple parallel channels that divide the vapor-air mixture into separate streams, each thermally coupled to a different portion of the cooling side of device 440, before recombining upstream of the mouthpiece 340. The heating air flow path 520 similarly admits of variation: ambient air can enter through side vents rather than top vents, or can be drawn through the lower vents 560 and expelled through the top vents 330, reversing the direction shown in FIG. 5.

The fan motor 420 and fan blades 430 are positioned near the top of the heating chamber in FIGS. 4-5, but other placements are feasible. For example, the fan assembly is positioned at the base of the heating chamber, drawing air downward through the top vents 330 and forcing it upward across the thermal coupling fins 470 before expelling it through side or top exhaust vents. In some examples, multiple smaller fans are distributed throughout the heating chamber rather than using a single centralized fan, improving airflow uniformity and reducing acoustic noise. The fan can operate at variable speeds controlled by a microcontroller that adjusts fan speed based on temperature sensors, user input, or battery charge level, optimizing thermal performance and power consumption across different operating conditions.

The container 460 shown in FIGS. 4-5 can be a discrete cartridge positioned adjacent to the heating chamber. This cartridge can be any type of container, such as an industry-standard 510-thread cartridge, a refillable cartridge, or any other type of container. For example, alternative container configurations include integrated reservoirs molded into the device housing, disposable pre-filled cartridges that snap into place without user refilling, or modular container assemblies that allow the user to swap between different substances or capacities. The container need not be positioned to the right of the heating chamber; in some examples, the container wraps partially or fully around the heating chamber, maximizing thermal contact area and improving heating uniformity. In other examples, multiple smaller containers are distributed throughout the device, each thermally coupled to a different portion of the heating side of device 440, allowing the user to vaporize multiple substances simultaneously or sequentially without cross-contamination.

The cooling air flow path 510 described in relation to FIG. 5 includes several transitions where the cross-sectional area of the airflow passage changes, providing additional cooling through expansion effects. For example, as the vapor-air mixture exits the intermediate air passage 540 and enters the downward passage along the airflow passage wall 550, the channel widens, allowing the vapor-air mixture to expand. This expansion reduces the pressure and temperature of the mixture according to Bernoulli and Venturi principles, supplementing the active cooling provided by the heating and cooling device 440. Similarly, the transition from the baffled airflow passage 450 into the mouthpiece flow tube 410 includes a widening section that allows further expansion and cooling before the mixture reaches the user's mouth. In some examples, the mouthpiece 340 itself includes an expanding exit geometry that reduces the velocity and temperature of the exiting vapor-air mixture, improving comfort during inhalation. The transition from the container 460 into the intermediate air passage 540 can also incorporate an expansion zone that allows the freshly vaporized substance to cool slightly before entering the main cooling pathway, reducing the thermal load on the heating and cooling device 440.

The heating approach used in the examples described herein addresses several limitations of conventional vaporizer technologies that use a heating coil in direct contact with the substance. Such conventional approaches suffer from poor thermal transfer, resulting in long heating times, uneven heating, and localized burning of the substance in contact with the coil, which degrades flavor and produces harmful byproducts. In contrast, the heating and cooling device 440 (such as a thermoelectric device based on the Peltier effect) warms the substance indirectly through thermal coupling fins 470 or other thermal interfaces, distributing heat more uniformly throughout the container 460. This indirect heating reduces the risk of localized overheating, preserves the flavor and chemical integrity of the substance, and provides faster, more controllable heating compared to resistive coils.

Given the complexity of managing multiple thermal zones and airflow paths, sensor-based feedback control improves performance and user experience. In some examples, temperature sensors are positioned at multiple locations, including within or adjacent to the container 460, along the cooling air flow path 510, within the heating chamber, and on the heating and cooling device 440 itself. A microcontroller receives temperature readings and adjusts the electrical power supplied to the heating and cooling device 440 to maintain target temperatures. For example, if the vapor-air mixture temperature exceeds a threshold, the microcontroller increases power to device 440 to enhance cooling; if the substance temperature falls below a target, the microcontroller increases heating power. Viscosity sensors or flow rate sensors detect the consistency of the substance within container 460, providing feedback that allows the microcontroller to adjust heating power to maintain optimal viscosity for vaporization. Fan speed is also controlled based on sensor feedback, increasing airflow when higher cooling capacity is needed and reducing airflow to conserve battery power when thermal loads are low. This adaptive control optimizes thermal performance, power consumption, and battery life across varying usage patterns and environmental conditions.

The baffled airflow passage 450 described in relation to FIGS. 4-5 includes internal structures that redirect the vapor-air mixture to increase turbulence and maximize thermal surface area contact with the cooling side of the heating and cooling device 440. In some examples, these internal structures comprise redirection vanes, which can be thin, angled plates or fins that extend into the airflow passage and deflect the vapor-air mixture toward the cooling surfaces. The redirection vanes can be oriented at various angles relative to the primary flow direction, creating swirling or zigzag flow patterns that increase the residence time of the vapor-air mixture within the passage. For example, a series of alternating vanes angled in opposite directions creates a serpentine flow path that forces the vapor-air mixture to contact the cooling surfaces multiple times before exiting the passage. In other examples, the redirection vanes are curved or helical, inducing rotational flow that improves convective heat transfer coefficients. The vanes can be formed integrally with the walls of the airflow passage or installed as separate components, and their spacing, angle, and geometry can be optimized to balance thermal performance against pressure drop and airflow resistance.

The mouthpiece 340 and mouthpiece flow tube 410 can incorporate venturi tube geometry to provide additional cooling through controlled expansion and acceleration of the vapor-air mixture. In some examples, the mouthpiece flow tube 410 includes a narrowed section positioned between the baffled airflow passage 450 and the exit opening of the mouthpiece 340. As the vapor-air mixture enters the narrowed section, its velocity increases and its pressure decreases according to the Venturi effect. Downstream of the narrowed section, the passage expands, allowing the vapor-air mixture to decelerate and further reduce in temperature as the pressure recovers. This expansion cooling supplements the active cooling provided by the heating and cooling device 440, reducing the final temperature of the vapor-air mixture delivered to the user. In an example, the narrowed section has a diameter 30-70% smaller than the upstream and downstream passages, creating a significant pressure drop and velocity increase. The expansion downstream of the narrowed section can be gradual (a diffuser geometry) or abrupt (a sudden expansion), depending on the desired balance between cooling effectiveness and pressure recovery. The venturi geometry also creates a localized low-pressure zone that can draw additional ambient air into the vapor-air mixture through auxiliary inlets, diluting the vapor concentration and further cooling the mixture.

In some examples, the device includes an auxiliary airflow passage that feeds ambient air into the main cooling air flow path 510 at one or more locations along its length. The auxiliary airflow passage provides a secondary source of cool air that mixes with the vapor-air mixture, diluting the vapor concentration and reducing the temperature through mixing. For example, an auxiliary passage connects the lower air vents 560 to the intermediate air passage 540, allowing ambient air to enter the cooling air flow path 510 upstream of the baffled airflow passage 450, reducing the thermal load on the heating and cooling device 440. In other examples, auxiliary passages connect to the airflow passage wall 550 or the baffled airflow passage 450 at multiple points, introducing ambient air incrementally along the flow path. The auxiliary passages can include one-way valves or flow restrictors that control the amount of auxiliary air admitted, allowing adjustment of vapor concentration and cooling rate.

In some examples, the device includes a liquid filtration module positioned within the cooling air flow path 510 through which the vapor-air mixture travels before reaching the user's mouth. The liquid filtration module removes particulates, condensed droplets, or undesired chemical species from the vapor-air mixture, improving the purity and quality of the inhaled vapor. The module includes a chamber containing a liquid filtration medium, such as water, glycerin, propylene glycol, or another liquid that selectively absorbs or traps contaminants. As the vapor-air mixture passes through or bubbles through the liquid filtration medium, particulates and water-soluble compounds dissolve or become trapped in the liquid, while the vapor phase continues through the module. For example, the liquid filtration module is positioned between the baffled airflow passage 450 and the mouthpiece flow tube 410, allowing the cooled vapor-air mixture to pass through the liquid medium immediately before delivery to the user. The liquid filtration medium is replaced or replenished periodically to maintain filtration performance.

While the examples in FIGS. 4-5 depict a single heating and cooling device 440, some configurations include a plurality of heating and cooling devices distributed throughout the inhalation device. For example, a first heating and cooling device is positioned to cool the intermediate air passage 540, a second device is positioned to cool the airflow passage wall 550, and a third device is positioned to cool the baffled airflow passage 450, allowing staged cooling of the vapor-air mixture as it travels through the cooling air flow path 510. The heating sides of the multiple devices are all thermally coupled to the same container 460, distributing the heating load across multiple thermal interfaces and improving heating uniformity. In some examples, the plurality of heating and cooling devices are arranged in series along the cooling air flow path 510, with each device extracting a portion of the thermal energy from the vapor-air mixture. In other examples, the devices are arranged in parallel, with the vapor-air mixture divided into multiple streams that each pass through a separate device before recombining. The use of multiple smaller devices rather than a single large device improves redundancy, allows finer thermal control, and reduces peak power demand on the battery.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, or in a different order. The order of steps presented are only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the disclosure being indicated by the following claims.

What is claimed is:

1. An inhalation device, comprising:
a heating and cooling device configured to extract heat from a first side of the device and expel heat from a second side of the device;
an airflow passage thermally coupled to the first side of the heating and cooling device;
a container thermally coupled to the second side of the heating and cooling device; and
an atomizer for thermally decomposing a substance within the container, such that the decomposed substance travels through the airflow passage.

2. The inhalation device of claim 1, wherein the heating and cooling device is a thermoelectric device.

3. The inhalation device of claim 1, wherein the container is thermally coupled to the second side of the device by a plurality of heat fins.

4. The inhalation device of claim 1, wherein the heating and cooling device is shaped such that the airflow passage and container are located on opposing sides of the inhalation device.

5. The inhalation device of claim 1, further comprising a plurality of heating and cooling devices.

6. The inhalation device of claim 1, wherein the heating and cooling device is shaped such that the heating and cooling device surrounds the airflow passage and the container surrounds the heating and cooling device.

7. The inhalation device of claim 1, wherein the airflow passage comprises a plurality of redirection vanes positioned within the airflow passage.

8. The inhalation device of claim 1, further comprising a fan positioned within a body of the inhalation device, wherein expelling heat from the second side of the device further comprises turning on the fan to produce air flow against the second side of the device.

9. The inhalation device of claim 1, further comprising a mouthpiece with an internal venturi tube that includes a narrowed section that expands before terminating in an exit port of the mouthpiece.

10. The inhalation device of claim 1, further comprising an auxiliary airflow passage that feeds air into the airflow passage.

11. The inhalation device of claim 1, wherein the airflow passage includes at least one portion shaped to cause fluid flow through the airflow passage to expand.

12. The inhalation device of claim 1, further comprising a thermal sensor positioned to determine the temperature of fluid flow through the airflow passage in at least one location.

13. The inhalation device of claim 1, further comprising a viscosity sensor positioned to determine a viscosity of fluid within the device.

14. The inhalation device of claim 1, wherein the heating and cooling device automatically adjusts a power input based on input received from a sensor in the inhalation device.

15. An inhalation device, comprising:
a heating and cooling device configured to extract heat from a first side of the device and expel heat from a second side of the device;
an airflow passage thermally coupled to the first side of the heating and cooling device;
a container thermally coupled to the second side of the heating and cooling device;
an atomizer for thermally decomposing a substance within the container, such that the decomposed substance travels through the airflow passage; and
a liquid filtration module configured such that the decomposed substance travels through the liquid in the filtration module.

16. The inhalation device of claim 15, wherein the heating and cooling device is a thermoelectric device.

17. A method of heating and cooling an inhalation device, comprising:
heating a substance within a container of the inhalation device, wherein at least a portion of the heating is performed by a heating and cooling device configured to extract heat from a first side of the device and expel heat from a second side of the device;
causing the substance to at least partially vaporize by application of a heating element; and
causing the vaporized substance to travel through an airflow passage thermally coupled to the first side of the heating and cooling device.

18. The method of claim 17, wherein the heating and cooling device is a thermoelectric device.

19. The method of claim 17, further comprising causing the vaporized substance to expand based on the airflow passage expanding in cross-sectional diameter.

20. The method of claim 17, further comprising turning on a fan positioned within a body of the inhalation device, wherein the fan causes heat to be expelled by producing airflow against the second side of the device.
